(19) United States
(12) Patent Application Publication
(10) Pub. No.: US 2010/0145154 A1
(43) Pub. Date: Jun. 10, 2010

(54) Rodent Cremaster Preparation Device for Intravital Microscopy

(75) Inventor: Pengxuan Liu, El Sobrante, CA (US)

Correspondence Address: BAYER HEALTHCARE LLC, Law & Patents, 800 Dwight Way, BERKELEY, CA, 94710, US

(73) Assignee: BAYER HEALTHCARE LLC, Tarrytown, NY (US)

(21) Appl. No.: 12/550,176

(22) Filed: Aug. 28, 2009

Related U.S. Application Data
(60) Provisional application No. 61/092,539, filed on Aug. 28, 2008.

(51) Int. Cl. A61B 1/32; G02B 21/30

(52) U.S. Cl. 600/235; 359/395

(57) ABSTRACT

Provided herein is a device useful for preparing a cremaster muscle of a specimen for intravital microscopy.

### REFERENCE TO RELATED APPLICATIONS

The present application claims benefit of priority to U.S. Provisional Application No. 61/092,539, filed Aug. 28, 2008 which is hereby incorporated by reference for all purposes.

### FIELD

Provided is a device useful for preparing and securing a rodentcremaster muscle for intravital microscopy.

### BACKGROUND

Intravital microscopy is considered a useful tool for in vivo investigations of dynamic microvascular regulation. Intravital microscopy requires an appropriate animal model, a molecular probe (usually fluorescent), a microscope equipped with a digital camera detection system, an image acquisition system, and a computer to process and analyze the data to extract parameters of interest.

The cremaster muscle is frequently used as the vascular window for the study of microcirculation in vivo. This muscle is a thin and translucent muscular tissue that surrounds a testis. The translucent nature of the muscle allows direct observation of blood cells circulating within the arterioles and venules by intravital microscopy.

For intravital microscopy, a cremaster muscle holder should allow the secure mounting of the muscle at physiological temperature. A common method for mounting the cremaster muscle of a living mouse for intravital microscopy is by using a tray containing a circular glass slide surrounded by a silicon gasket in the center of the tray. With an anesthetized mouse fixedly secured to the tray, one of the testis is exteriorized and placed on a circular glass slide. The muscle is then splayed and secured on top of the glass slide by placing needles through the muscle and into a silicon gasket. This setup results in a small, approximately 1 cm², observation area for intravital microscopy. Physiological buffer warmed to a predetermined temperature is then superfused over the cremaster muscle and the waste buffer is collected in the tray.

### DESCRIPTION OF THE DRAWINGS

FIG. 1 depicts a schematic of a top-down view of a surgery tray of the cremaster muscle device for intravital microscopy. Measurements are provided in centimeters (cm).

FIG. 2 depicts a schematic of a side view of a surgery tray of the cremaster muscle device for intravital microscopy. Measurements are provided in cm.

FIG. 3 depicts a schematic of a top-down view of a temperature-controlled cremaster muscle mount. Measurements are provided in cm.

FIG. 4 depicts a schematic of a side view (a), a front view (b) and a back view (c) of a temperature-controlled cremaster muscle mount. Measurements are provided in cm.

### SUMMARY

Provided is a device useful for securing a cremaster muscle of a specimen, particularly a rodent, during intravital microscopy comprising: a) a surgery tray, b) immobilizers, and c) a temperature-controlled cremaster muscle mount, wherein the mount comprises a temperature sensor, a heating element, a viewing plate, a well formed by a gasket for physiological buffer, and a plurality of hooks and sutures, wherein the mount is positioned onto the surgery tray to hold the cremaster muscle of the specimen and wherein the hooks are positioned inside the well and are suitable for spreading the cremaster muscle, whereby the cremaster muscle is viewable under a microscope.

Also provided is a mounting device useful for securing a cremaster muscle of a specimen during intravital microscopy comprising: a) a plate, b) a plate mount, c) a plurality of stand-offs, d) a temperature sensor, e) a heating element, f) a gasket, wherein the gasket creates a well, and g) a plurality of hooks and sutures. Further, the present application provides for a method of using this device, wherein the cremaster muscle is positioned on the plate within the gasket and is stretched to a thin layer using the hooks and sutures.

Further, a method of preparing a cremaster muscle for intravital microscopy is provided, the method comprising: a) providing a specimen, b) placing the specimen on a surgery tray, c) immobilizing the specimen using immobilizers situated in specific positions on the tray, d) isolating the specimen's cremaster muscle from the scrotum, e) positioning the cremaster muscle onto a temperature-controlled cremaster muscle mount, f) inserting hooks attached to sutures positioned in a circular array into the cremaster, and g) spreading the cremaster muscle using the hooks and sutures to a thin layer.

### DETAILED DESCRIPTION OF THE INVENTION

It is to be understood that this invention is not limited to the particular methodology, protocols, cell lines, animal species or genera, constructs, and reagents described and as such may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural reference unless the context clearly dictates otherwise. Thus, for example, reference to “a hook” is a reference to one or more hook and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are hereby incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### Device for Intravital Microscopy of Cremaster Muscle

For intravital microscopy of the cremaster muscle, the muscle is isolated from the scrotum and is spread thinly over a surface to allow viewing of arterioles and venules. It is necessary for the cremaster muscle to be held securely in place for optimal viewing under the microscope. In addition, it is necessary for the cremaster muscle to be immersed in physiological buffer and for the specimen to be kept at a desired temperature, e.g. approximately 35° C., to provide the optimal conditions for the blood to flow.

Thus, provided is a device useful for securing the cremaster muscle, useful for immersing the cremaster in physiological buffer, and useful for maintaining a desired temperature of the specimen. The present application provides for a device useful for securing a cremaster muscle of a specimen during intravital microscopy comprising: a) a surgery tray, b) immobilizers, and c) a temperature-controlled cremaster muscle mount, wherein the mount comprises a temperature sensor, a heating element, a viewing plate, a well formed by a gasket for physiological buffer, and a plurality of hooks and sutures, wherein the mount is positioned onto the surgery tray to hold the cremaster muscle of the specimen and wherein the hooks are positioned inside the well and are suitable for spreading the cremaster muscle, whereby the cremaster muscle is viewable under a microscope.

In some embodiments, the device further comprises a micropump and tubing to circulate physiological buffer over the cremaster. In some embodiments, the micropump is placed in a container containing physiological buffer with an output and input tube located within the well of the mount, thereby recirculating the buffer. In some embodiments, the micropump provides fresh physiological buffer and waste buffer is pumped into a waste container. In some embodiments, the physiological buffer is heated to a predetermined temperature. This predetermined temperature is the typical average body temperature of the specimen. In some embodiments, the temperature of the buffer is about 35° C.

In some embodiments, the device further comprises a heat pad which is useful for maintaining the specimen at the predetermined temperature.

In some embodiments, the specimen is one that is suitable for intravital microscopy. In some embodiments, the specimen is a mammal. In some embodiments, that mammal is a rodent, for example a mouse or a rat.

### Surgery Tray and Immobilizers

A surgery tray provides a platform on which the specimen is prepared for intravital microscopy. The tray provides a place for run-off bodily fluids or physiological buffer to collect. In some embodiments, the surgery tray is fabricated from a material selected from the group consisting of a plastic, a polymer, a glass, a metal or a combination thereof. In some embodiments, the surgery tray features additional body boards for placing below the specimen thereby raising the specimen from the tray.

In some embodiments, the surgery tray comprises a magnetic element for variable positioning and fixing of the temperature-controlled cremaster muscle mount to the tray.

In some embodiments, the immobilizers comprise pegs and bands. In some embodiments, the pegs are positioned on the surgery tray such that they are useful for immobilizing the ilium and rear legs. In some embodiments, the bands are useful for immobilizing the head and body. In some embodiments, the bands are made of an elastic material, such as rubber bands or flexible tubing (e.g. silicon or rubber tubing).

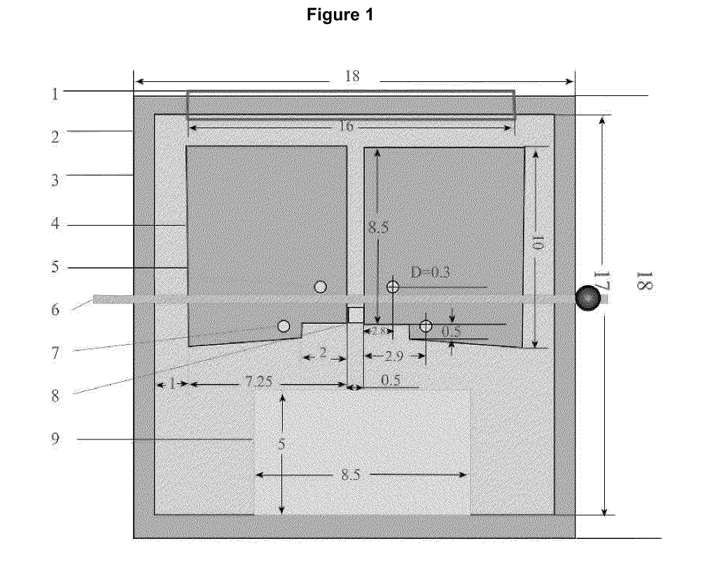

Referring now to FIG. 1, shown from above is a schematic of the surgery tray (3) featuring the magnetic element (9), and the immobilizers (1, 5, 6, and 7). To secure the head, an elastic band (1) is used. For example, this band can be secured below the two front teeth of the specimen, thereby pulling the head down and back providing easy access to the specimen's throat and jugular vein. Thus, providing optimal positioning for injection of a molecular probe.

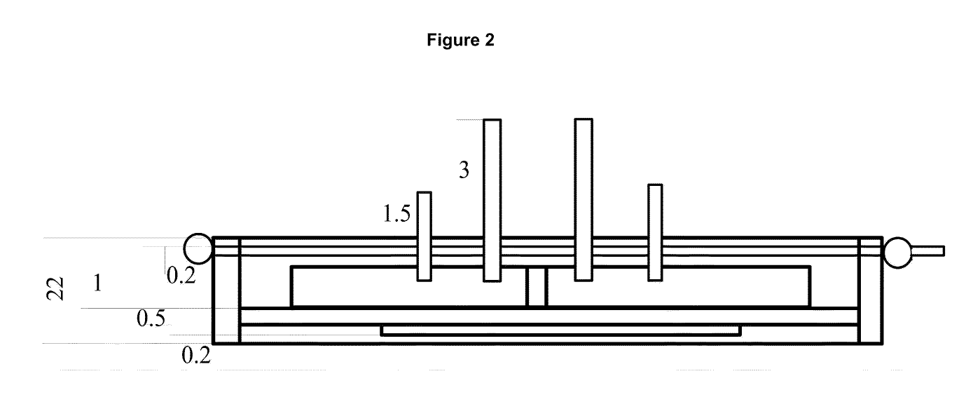

To secure the body firmly to the tray, a second elastic band (6) is positioned above the belly of the specimen. Pegs (5 and 7) are used to secure the ilium of the specimen and the feet of the specimen. Also shown in the schematic are two body boards (4) for raising the height of the specimen from the tray. The boards also allow the tail to be positioned between the boards so that it is not in the way of the area of interest. In this schematic, a tail fixer (8) is used to push the tail through the gap formed between the boards. FIG. 2 provides a schematic of the surgery tray from the front.

### Temperature-Controlled Cremaster Muscle Mount

Also provided is a mounting device useful for securing a cremaster muscle of a specimen during intravital microscopy comprising: a) a plate, b) a plate mount, c) a plurality of stand-offs, d) a temperature sensor, e) a heating element, f) a gasket, wherein the gasket creates a well, and g) a plurality of hooks and sutures. Further, the present invention provides for a method of using this device, wherein the cremaster muscle is positioned on the plate within the gasket and is stretched to a thin layer using the hooks and sutures.

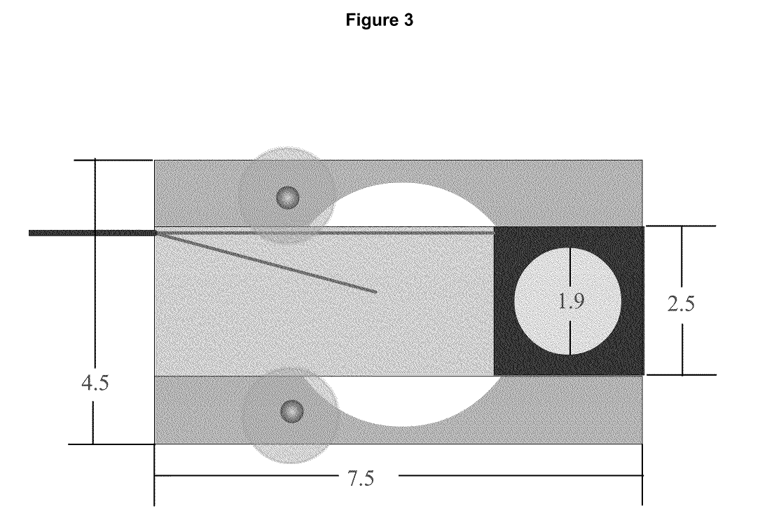

Referring now to FIG. 3, shown is a schematic of the mounting device. The mounting device comprises a plate (101), a plate mount (102), a set of stand-offs (107), a gasket (103), a well (104), a heating element (105), and a temperature sensor (106). The mounting device is positioned on the surgery tray such that the cremaster muscle can be secured within the well (104). The heating element and temperature sensor are to maintain the specimen's cremaster at a desired and predetermined temperature.

In some embodiments, the plate (101) is a glass slide. In some embodiments, the plate mount (102) is fabricated from a material selected from a plastic, metal or glass. The plate mount can be fabricated for example by mold forming or by direct machining.

In some embodiments, the stand-offs (107) comprise magnetic elements at their base so that they can be secured and attached to the magnetic element of the surgery tray. In some embodiments, the stand-offs are adjustable in height (e.g. a screw type height adjustment).

The gasket (103) forms a well (104) in which the cremaster muscle and physiological buffer are placed. In some embodiments, the gasket is a material selected from a rubber, silicone, plastic, metal or glass. In some embodiments, a plurality of hooks and sutures are used to secure the cremaster muscle. Based upon the schematic provided, the hooks are located inside the well and are connected to sutures that run through the gasket to the outside of the well. The hooks are then pierced through the cremaster muscle and when the sutures are pulled outwards away from the center of the well, the cremaster muscle is spread within the well. For example, the sutures may be a 7-0 nylon suture having a diameter of 50 μm. Other suitable materials may also be used, for example metal wire, etc.

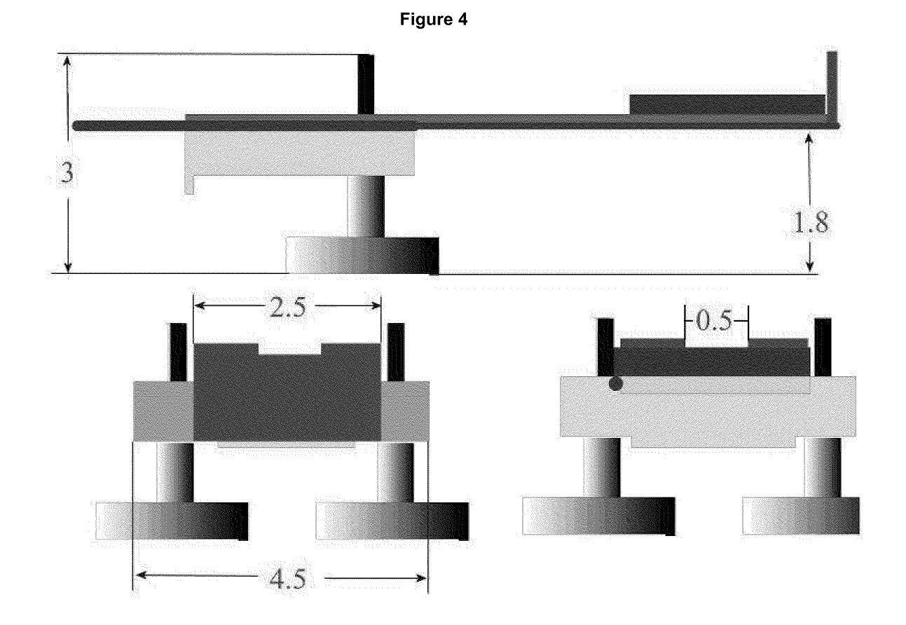

FIG. 4 provides alternate views of the schematic of the mounting device.

### Method of Preparing Cremaster Muscle

A method of preparing a cremaster muscle for intravital microscopy is provided, the method comprising: a) providing a specimen, b) placing the specimen on a surgery tray, c) immobilizing the specimen using immobilizers situated in specific positions on the tray, d) isolating the specimen's cremaster muscle from the scrotum, e) positioning the cremaster muscle onto a temperature-controlled cremaster muscle mount, f) inserting hooks attached to sutures positioned in a circular array into the cremaster, and g) spreading the cremaster muscle using the hooks and sutures to a thin layer.

### EXAMPLES

### Example 1

### Preparation of Cremaster

Briefly, mice were anesthetized with ketamine-xylazine injected intraperitoneally and the cremaster muscle sac was isolated from the scrotum and placed on a histological glass slide. A longitudinal incision was made in the ventral surface of the muscle and the muscle was gently pulled and spread over the well. The preparation was irrigated with physiological saline solution (PSS), 33-34° C., pH 7.4, composed of (in mM) 131.9 NaCl, 4.7 KCl, 2.2 CaCl₂, 1.2 MgSO₄ and 20.0 NaHCO₃ bubbled continuously with 5% CO₂/95% N₂ gas. The mouse was kept warm with a heating pad and the cremaster was kept warm by the heating element.

All publications and patents mentioned in the above specification are incorporated herein by reference. Various modifications and variations of the described methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the above-described modes for carrying out the invention which are obvious to those skilled in the art are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

1. A device useful for securing a cremaster muscle of a specimen during intravital microscopy comprising:
a) a surgery tray,
b) immobilizers, and
c) a temperature-controlled cremaster muscle mount, wherein the mount comprises a temperature sensor, a heating element, a viewing plate, a well formed by a gasket for physiological buffer, a plurality of stand-offs, and a plurality of hooks and sutures,
wherein the mount is positioned onto the surgery tray to hold the cremaster muscle of the specimen and wherein the hooks are positioned inside the well and are suitable for spreading the cremaster muscle, whereby the cremaster muscle is viewable under a microscope.

2. The device of claim 1 further comprising a micropump and tubing, wherein the micropump and tubing circulate physiological buffer over the cremaster muscle within the well.

3. The device of claim 1 further comprising a heating pad.

4. The device of claim 1, wherein the specimen is a mammal.

5. The device of claim 4, wherein the mammal is a rodent.

6. The device of claim 1, wherein the surgery tray is fabricated from a material selected from the group consisting of a plastic, a polymer, a glass, a metal or a combination thereof.

7. The device of claim 1, wherein the surgery tray comprises a magnetic element for variable positioning and fixing of the temperature-controlled cremaster muscle mount to the tray.

8. The device of claim 1, wherein the immobilizers comprise pegs and bands, wherein the pegs are useful for immobilizing the ilium and rear legs and wherein the bands are useful for immobilizing the head and body.

9. The device of claim 1, wherein the well for physiological buffer surrounds a region of the viewing plate to immerse the cremaster muscle in buffer.

10. The device of claim 1, wherein the hooks are fabricated from wire.

11. A mounting device useful for securing a cremaster muscle of a specimen during intravital microscopy comprising:
a) a plate,
b) a plate mount,
c) a plurality of stand-offs,
d) a temperature sensor,
e) a heating element,
f) a gasket, wherein the gasket creates a well, and
g) a plurality of hooks and sutures.

12. The device of claim 11, wherein the plate is glass.

13. The device of claim 11, wherein the plate mount is a material selected from the group consisting of plastic, metal, or glass.

14. The device of claim 11, wherein the stand-off is a material selected from the group consisting of plastic, metal, or glass.

15. The device of claim 11, wherein the stand-off is adjustable in height.

16. The device of claim 11, wherein the stand-off comprises a means for attachment to a tray.

17. The device of claim 16, wherein the means for attachment to the tray is magnetic.

18. The device of claim 11, wherein the gasket is a material selected from the group consisting of a rubber, silicone, plastic, metal, or glass.

19. The device of claim 11, wherein the hooks are fabricated from wire.

20. The device of claim 11, further comprising a micropump and tubing, wherein the micropump and tubing circulate physiological buffer over the cremaster muscle within the well.

21. A method of using the device of claim 11, wherein a cremaster muscle of a specimen is positioned on the plate within the gasket and is stretched to a thin layer using the hooks.

22. A method of preparing a cremaster muscle for intravital microscopy comprising the steps of:
a) providing a specimen,
b) placing the specimen on a surgery tray,
c) immobilizing the specimen using immobilizers situated in specific positions on the
tray,
d) isolating the specimen's cremaster muscle from the scrotum,
e) incising and positioning the cremaster muscle onto a temperature-controlled cremaster muscle mount,
f) inserting hooks attached to sutures positioned in a circular array into the cremaster, and
g) spreading the cremaster muscle using the hooks and sutures to a thin layer.
